(19) United States
(12) Patent Application Publication
(10) Pub. No.: US 2005/0242301 A1
(43) Pub. Date: Nov. 3, 2005

(54) Targeted radiation treatment using a spectrally selective radiation emitter

(75) Inventor: Brian N. Pierce, Chico, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Advanced Light Technologies, LLC, a corporation of the state of California, Chico, CA (US)

(21) Appl. No.: 11/033,410

(22) Filed: Jan. 10, 2005

Related U.S. Application Data
(60) Provisional application No. 60/536,055, filed on Jan. 12, 2004.
(60) Provisional application No. 60/571,236, filed on May 13, 2004.

(51) Int. Cl. G03C 5/16

(52) U.S. Cl. 250/492.2

(57) ABSTRACT

Radiation from a spectrally broad radiation source is reduced to radiation of limited spectral range with high efficiency by an emitter that includes a radiation source and a multilayer optical coating that reflects radiation of certain wavelengths back toward the source, allowing other wavelengths to pass. The multilayer optical coating provides a high efficiency reflectance, thereby minimizing loss of radiation energy despite limiting the escaping energy to one or more narrow selected wavelength bands. The resulting radiation is useful in treating a host to destroy or deactivate undesirable pathogens, cells, or tissues.

### CROSS-REFERENCE TO RELATED APPLICATIONS

This application is related to U.S. Provisional Patent Applications Nos. 60/536,055, filed Jan. 12, 2004, and 60/571,236,filed May 13, 2004, and claims all benefits legally capable of being offered by both provisional patent applications. The entire contents of both provisional patent applications are incorporated herein by reference.

### BACKGROUND OF THE INVENTION

The irradiation of materials with electromagnetic waves at selected wavelengths for various purposes is disclosed in International Publication No. WO 02/091394 A1 (entitled “Differential Photochemical and Photomechanical Processing;” Advanced Light Technology, LLC, applicant; publication date Nov. 14, 2002), published under the Patent Cooperation Treaty, in U.S. Pat. No. 5,820,820 (entitled “Method of Thermally and Selectively Separating Water and/or Solvents From Solids Under Vacuum Utilizing Radiant Heat;” Brian N. Pierce, applicant; issue date Oct. 13, 1998), and in U.S. Pre-Grant Patent Publication No. US 2004/0236267 A1 (entitled “Disinfection, Destruction of Neoplastic Growth, and Sterilization by Differential Absorption of Electromagnetic Energy;” Brian N. Pierce, applicant; publication date Nov. 25, 2004). These and all other patents and published patent applications cited throughout this specification are hereby incorporated herein by reference in their entirety for all purposes legally capable of being served thereby.

Each of the above disclosures cites the value of focusing the wavelength of the radiation to a narrow band to achieve specified results in the materials without undesired side effects. Among the results achieved by this focused irradiation are disinfection, disinfestation, sterilization, denaturation, and dehydration of the material, and destroying or disrupting a specified component of the material. The focusing allows the result to be achieved without harm to, or other conversion of, the remainder of the material. The appropriate wavelength for the desired effect is determined by first determining the capacity of the material as a whole to withstand irradiation without damage, i.e., the range of temperature or irradiation to which the material can be exposed without damage or loss of function, determining the wavelength of radiation that will be preferentially or selectively absorbed by the component sought to be destroyed or converted, and then exposing the entire material to radiation at that wavelength. Among the materials that can be treated in this manner are organic matter including foodstuffs, inorganic matter including medical devices and implants, and living matter including tissue, organs and organisms.

The disclosures cited above list various sources of radiation, including lasers, gas discharge tubes, black body sources, infrared light sources, and other devices that produce either broad or narrow emission spectra. In each case, however, the scope of application of the procedure is limited since focused irradiation can only be performed at wavelengths that are supplied by commercially available focused radiation emitters, unless the process can tolerate radiation of a relatively broad spectrum. The present invention overcomes these limitations by providing a way of producing focused irradiation in a manner that is independent of the energy source, thereby broadening the applicability of the treatment and the versatility of the equipment.

### SUMMARY OF THE INVENTION

The present invention resides in an improvement in the procedures disclosed in the documents cited above, by the use of a spectrally selective radiation emitter as the source of electromagnetic radiation. The radiation emitter utilizes a broad-spectrum source of electromagnetic radiation disposed inside a housing in combination with a multilayer optical coating that allows radiation of one or more selected wavelengths or wavelength bands to pass while reflecting radiation of other wavelengths back from the coating to cause the reflected radiation to be retained in the interior of the housing. The multilayer coating, which functions in a manner known in the art, contains a multitude of layers of optical material of alternating refractive index values, the layers having optical thicknesses selected to produce constructive interference between reflected radiations from different layer pairs. Accordingly, the wavelength of the radiation escaping from the housing is controlled by the multilayer coating rather than the radiation source. An advantage of the emitter is that unwanted radiation is reflected back toward the source to be regenerated until it is converted to radiation of the desired wavelength(s). As a result, there is at most a minimal loss of energy by absorption within the emitter. The radiative emission leaving the emitter is thus of a controlled wavelength and bandwidth and is produced at high efficiency.

In certain embodiments of the invention the multilayer coating is a composite stack whose layers form two or more segments, arranged either at discrete depths within the stack or superimposed over each other in a common region of the stack, each segment configured to reflect radiation of different wavelength ranges. Segments can be combined to bracket a wavelength or wavelength range of interest, i.e., to reflect radiation at wavelengths both above and below desired wavelength(s) while allowing only radiation at the desired wavelength(s) to escape the housing for productive use. Segments can also be combined to broaden the range of reflected wavelengths, thereby offering additional flexibility in the choice of the desired radiation.

In certain preferred embodiments as well, the emitter contains a total reflector to direct radiation leaving the housing in a selected direction.

Other objects, advantages, features, and variations will be apparent from the description that follows.

### BRIEF DESCRIPTION OF THE DRAWINGS

FIG. 1 is a plot of radiation intensity vs. wavelength curves for various gray body radiation sources.

FIG. 2 is a cross section of a flat panel emitter representing one example of an implementation of the present invention.

FIG. 3 is a cross section of either a cylindrical or spherical emitter representing another example of an implementation of the present invention.

FIG. 4 is a cross section of an emitter with a parabolic profile representing a third example of an implementation of the present invention.

FIG. 5 is a cross section of an emitter formed from a combination of emitters with parabolic profiles, representing a fourth example of an implementation of the present invention.

### DETAILED DESCRIPTION OF THE INVENTION AND PREFERRED EMBODIMENTS

Radiation sources suitable for use in the present invention include any device or component that emits electromagnetic radiation extending over a continuous spectrum, broad or narrow, of wavelengths. Useful spectra include those from the ultraviolet region to the far infrared region or any portion of or between these regions. Preferred sources are blackbody and graybody emitters whose emissions are based on temperature, rather than stimulated emission sources such as lasers. Blackbody and graybody emitters are useful for wavelengths in the visible or near infrared region, and in general for applications requiring wavelengths of about 6 microns or less.

Blackbody and graybody emitters are hot body sources, and solid hot body sources are particularly preferred. Virtually any body will emit electromagnetic energy over a wide spectral range when heated. The portion of this energy that resides in the infrared region is often called thermal radiation. The power of the thermal radiation and its spectral composition and distribution are determined by the properties of the body and the temperature to which the body is heated. The radiant flux rises rapidly with increasing temperature while the spectral composition shifts to shorter wavelengths. A fundamental property of thermal radiation is that radiative energy emitted by the body is derived from the thermal energy of the body itself. As it radiates energy, the body loses thermal energy and cools. To maintain the temperature of the body, the depleted thermal energy must be replaced by any energy source, including electrical currents and other stimulated emission devices, excitation, conduction, convection, or radiation from other bodies. Among the advantages of the present invention is that much of the depleted thermal energy is restored by radiation from the body itself that is outside of the desired wavelength range and reflected back to the body by the multilayer optical coating.

In applications where radiation in the infrared region is desired, a wide range of radiation sources can be used, extending from those emitting at high temperature and short wavelength to those emitting at low temperature and long wavelength. These emitters can be constructed from virtually any material that meets or exceeds thermal requirements and has the desired emissive qualities. Short wave emitters require a high temperature source and must be fabricated from materials that can maintain these high temperatures. All of the various types of commercially available infrared emitters emit energy over a wide spectral band of wavelengths. Examples are incandescent lights, heat lamps, resistance heaters, and gas and electric ceramic emitters. Blackbody emitters are those that function as ideal radiators emitting all energy, offering high power and wide spectral range at good efficiency. Gray body emitters are non-ideal, emitting energy in a selective manner and at less energy at any given wavelength than a blackbody.

The ratio of the energy radiated by a substance at a given temperature to the energy radiated by a blackbody at the same temperature is the emissivity ε. The emittance W is the power radiated per unit area of a radiating surface. Blackbody emittance varies with temperature and wavelength, and the relevant mathematical relations are expressed as Planck's Law, the Wien Displacement Law, and the Stefan-Boltzmann Law.

Planck's Law, expressed by Equation I below, governs the intensity of radiation emitted by a unit surface area in a fixed direction as a function of wavelength and temperature:

$${W}_{λ b} = \frac{2 π {hc}^{2}}{{λ}^{5} ( {ⅇ}^{hc / λ kT} - 1 )} \times {10}^{- 6} \qquad ( I )$$

where W_{λb}=the blackbody spectral radiant emittance at wavelength λ
c=3×1010 cm/sec (the speed of light)
h=6.625×10⁻²⁷ erg/sec (Planck's constant)
k=1.38×10⁻¹⁶ erg/K (Boltzmann's constant)
T=temperature in degrees K of the blackbody
λ=wavelength in microns
   Planck's Law establishes an intensity vs. wavelength distribution at a given temperature, the distribution including a peak at a certain wavelength. As the temperature rises, the peak shifts to shorter wavelengths and the area under the curve beneath the peak expands.

The Wien Displacement Law, expressed by Equation II below, expresses the wavelength having the maximum intensity as a function of temperature, showing the shift to shorter wavelengths as the temperature increases:

$${λ}_{max} = \frac{3 \times {10}^{7}}{T} \qquad ( II )$$

where λₘₐₓ=the wavelength in Angstroms of the emission having the maximum intensity
T=temperature in degrees K of the blackbody

The Stefan-Boltzmann Law, expressed by Equation III below, provides the total emittance E of the blackbody over all wavelengths, showing the growth in the height of the curve as the temperature increases:
E=σT⁴ (III)
where σ is the Stefan-Boltzmann constant 5.6705×10⁻⁵ erg/(cm²·T⁴) and T is the temperature of the blackbody in degrees K.

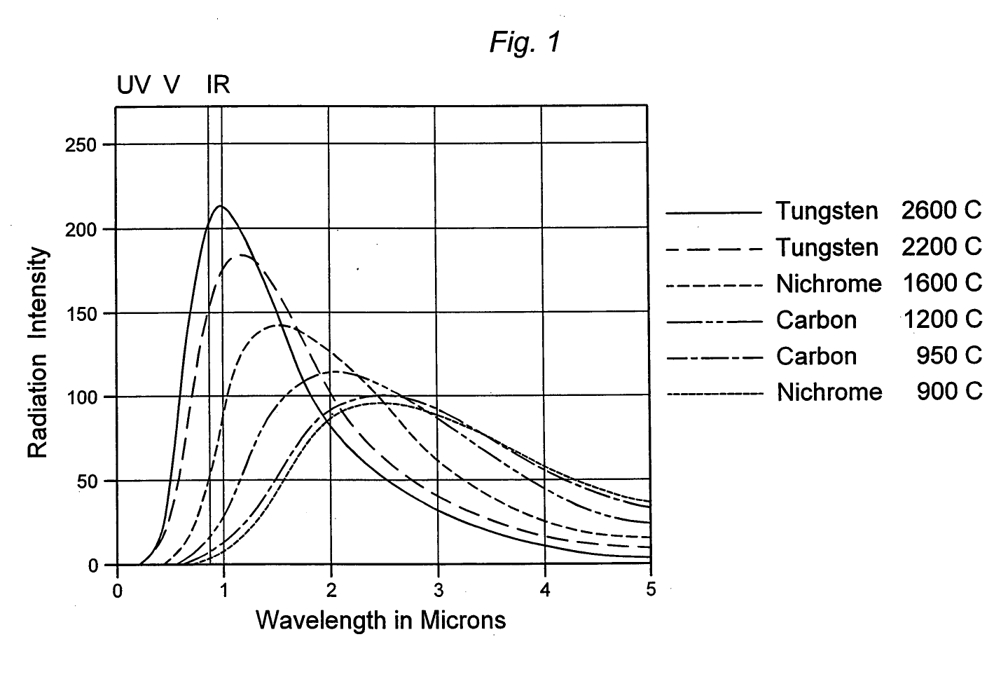

Gray body sources are likewise characterized by curves of radiation intensity vs. wavelength, which vary with the temperature of the gray body. Three examples of gray body materials are shown in FIG. 1 together with the intensity vs. wavelength curve for each material at each of two temperatures. The plot demonstrates that as the temperature drops the peak broadens and its maximum shifts to a lower intensity and a higher wavelength.

The multilayer optical coating utilizes principles known in the art and described for example in U.S. Pat. No. 4,663,557 (“Optical Coatings for High Temperature Applications,” Martin, R. L., et al., issued May 5, 1987), U.S. Pat. No. 6,451,414 B1 (“Multilayer Infrared Reflecting Optical Body,” Wheatley, J. A., et al., issued Sep. 17, 2002), U.S. Pat. No. 6,534,903 B1 (“Broad Spectrum Reflective Coating for an Electric Lamp,” Spiro, C. L., et al., issued Mar. 18, 2003), U.S. Pat. No. 6,567,211 B1 (“Dual-Band Millimeter-Wave and Infrared Anti-Reflective Coatings,” Dolezal, F. A., et al., issued May 20, 2003), U.S. Pat. No. 6,627,503 B2 (“Method of Forming a Multilayer Dielectric Stack,” Ma, Y., et al., issued Sep. 30, 2003), U.S. Pat. No. 6,668,111 B2 (“Optical Microcavity Resonator Sensor,” Tapalian, H. C., et al., issued Dec. 23, 2002), and U.S. Pat. No. 5,127,018 (“Helium-Neon-Laser Tube with Multilayer Dielectric Coating Mirrors,” Akiyama, Y., issued Jun. 30, 1992).

In the coating, adjacent layers differ in refractive index by differentials that alternate between positive and negative. The coating thus includes layers of high refractive index material alternating with layers of low refractive index material such that the differential between a pair of adjacent layers is positive while the differential between another pair of adjacent layers that has one layer in common with the first pair is negative. Reflection of incident radiation occurs at each interface as the incident radiation approaches the interface from the low-to-high direction. The wavelength at which peak reflectance occurs at a given low-to-high interface and the width of the peak reflectance curve vary with the ratio of refractive indices at that interface. Accordingly, by using known relationships, those skilled in the art can select the appropriate material pairs to achieve reflection at specified wavelengths or wavelength ranges. In most cases it is anticipated that a high-to-low refractive index ratio of at least about 1.3 will be used. Likewise, the high refractive index layers in most cases will have a refractive index equal to or greater than 2.1, preferably from about 2.1 to about 2.7, while low refractive index layers in most cases will have a refractive index equal to or less than 1.8, preferably from about 1.3 to about 1.8. Examples of materials suitable for use as the high refractive index layers are titanium oxides, zirconium oxides, manganese oxide, zinc sulfide, chromium oxide, zinc selenide, niobium oxide, indium oxide, and tantalum oxide. Examples of materials suitable for use as the low refractive index layers are gallium nitride, aluminum oxides, silicon oxides, calcium fluoride, magnesium fluoride, barium fluoride, cryolite (Na₃AlF₆), cerium fluoride, lanthanum fluoride, lead fluoride, neodymium fluoride, thorium fluoride, yttrium fluoride, and tungsten oxide. Materials with refractive indices between 1.8 and 2.1 can be used as either the high or low refractive index materials, depending on the materials with which they are paired. Examples of materials with refractive indices in this intermediate range are tin oxide, aluminum nitride, antimony oxide, yttrium oxide, silicon monoxide, cerium oxide, and hafnium oxide. In general, preferred materials for use as the high refractive index layers are zinc sulfide, zinc selenide, and titanium dioxide, while preferred materials for use as the low refractive index layers are cryolite, magnesium fluoride, and silicon dioxide. The combination of titanium dioxide and silicon dioxide is particularly preferred.

In accordance with known technology, the term “constructive interference” denotes the effect that occurs when reflected radiations from different low-to-high refractive index interfaces are in phase with each other and thereby reinforce rather than cancel each other. This is achieved by using quarter-wave layers, i.e., layers in which the optical thickness of each layer (the product of the actual thickness and the refractive index) is equal to, or a multiple of, one-fourth of the wavelength of the radiation sought to be reflected. Constructive interference is to be distinguished from “destructive interference” in which superimposed radiations are 180 degrees out of phase and cancel each other. Constructive interference occurs when twice the distance between reflecting surfaces (i.e., the combined distance traveled by the incident direction and the reflected direction before being superimposed over earlier reflected radiation) is an integer number of wavelengths divided by the refractive index. Destructive interference occurs when twice the distance between reflecting surfaces is one-half of the wavelength greater or less than an integer number of wavelengths divided by the refractive index by. The combined layers are commonly referred to as a “stack,” and since no single interface will produce total reflection, the greater the number of high and low refractive index layer pairs in the stack the greater the reflection from the stack. The width of the reflectance curve, i.e., the range of wavelengths that will be reflected by the stack, is determined by the refractive index ratio, as noted above, but can be increased by combining two or more stacks centered at different wavelengths or by slightly varying the layer thickness within a stack to form a layer thickness gradient within the stack.

The number of layer pairs within the stack or within a segment of the stack for stacks that contain two or more segments can vary, but as noted above, the total reflection from the stack will increase with the number of layer pairs. For most applications, it is contemplated that the stack, or any single segment of a multi-segment stack, will contain at least 3 pairs, or anywhere from 3 to 1,000 pairs, preferably from 4 to 50 pairs, and most preferably from 5 to 20 pairs. The number of pairs in one segment may differ from the number in another segment. In a presently contemplated embodiment, the emitter is designed for a single target wavelength, and the stack contains two segments, one reflecting radiation at wavelengths that are shorter than, and the other reflecting radiation at wavelengths that are longer than, the target wavelength. The number of pairs in the short-wavelength segment is 6 or 7 while the number of pairs in the long-wavelength segment is 10 or 11. In another presently contemplated embodiment, the emitter is designed for two target wavelengths, or two narrow bands at separate portions of the spectrum. The appropriate stack for this emitter is one that contains three or more segments to bracket (i.e., to reflect back all radiation except) the two target wavelength bands. The segments in a stack containing two or more segments can be positioned at discrete depths within the stack without physically overlapping, or may overlap within the stack to form a bi-modal or multi-modal stack. Arrangements of this type are known in the art and are shown in some of the patents cited above.

Aside from the multilayer optical coating or stack, certain embodiments of the invention further contain a total reflector, which is a layer or surface that reflects radiation from the source at all wavelengths, directing the radiation in a specified direction, or at a limited angle or a limited solid angle. The term “total reflector” is used herein to denote surfaces that reflect substantially all incident radiation, recognizing that true total reflection is an ideal that is not fully reached by any material. For a total reflector used in the present invention, the optimal direction of reflection will vary with the application or material to be treated. A variety of applications and materials to be treated are disclosed in the documents cited in the first paragraph under the heading “BACKGROUND OF THE INVENTION” above. The total reflector reflects radiation at all wavelengths and does not fully surround or envelop the radiation source, leaving a window or spatial region through which radiation can pass. The total reflector can be a further layer in the multilayer optical coating, i.e., an outer layer thereof, or a surface separate from the multilayer optical coating, i.e., on another portion of the housing. Any common reflective material that passes no radiation (or substantially no radiation) can be used. Metallic reflectors are examples.

Emitters of the present invention can also contain a polarizing coating on the output side of the emitter or of the housing. The polarizing coating will be arranged such that light from within the emitter will pass through the coating before fully escaping the housing. The coating can either be one layer of the multilayer optical coating or an additional coating forming the outermost layer.

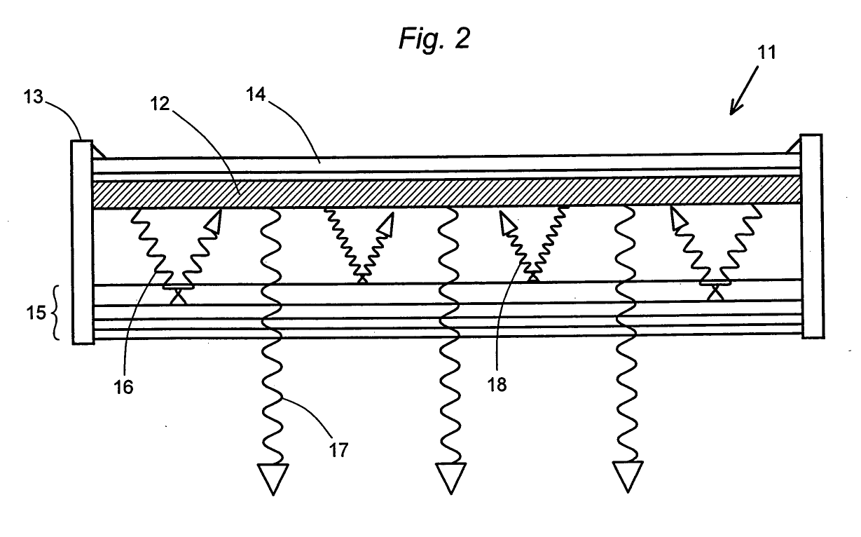

Emitters for use in the practice of the present invention can assume any of a variety of physical shapes and configurations, as can the arrangement of the radiation source and the multilayer optical coating components of the emitters. Such shapes include, but are not limited to, a flat panel, a parabola, a sphere, and an ellipse. Examples are shown in FIGS. 2, 3, 4 and 5.

The emitter of FIG. 2 is a flat panel emitter 11 shown in cross section. The radiation source 12 is a flat panel contained in a housing or envelope 13 with a total reflector 14 on one side of the source and a stack of multilayer optical coatings 15 on the other side of the source. The source is a blackbody or gray body source activated or energized by external means (not shown) that are conventional in nature. As is typical of black or gray body sources, the source emits radiation of a wide range of wavelengths 16, 17, 18 in all directions. The total reflector 14 prevents radiation from escaping the emitter at one side of the source 12 (the uppermost side in the view shown in the Figure). The multilayer optical coatings 15 collectively reflect radiation back to the source 12, with the benefit of constructive interference, at wavelengths both shorter 16 and longer 18 than the desired wavelength band 17, and allow radiation at the desired wavelength band 17 to leave the emitter.

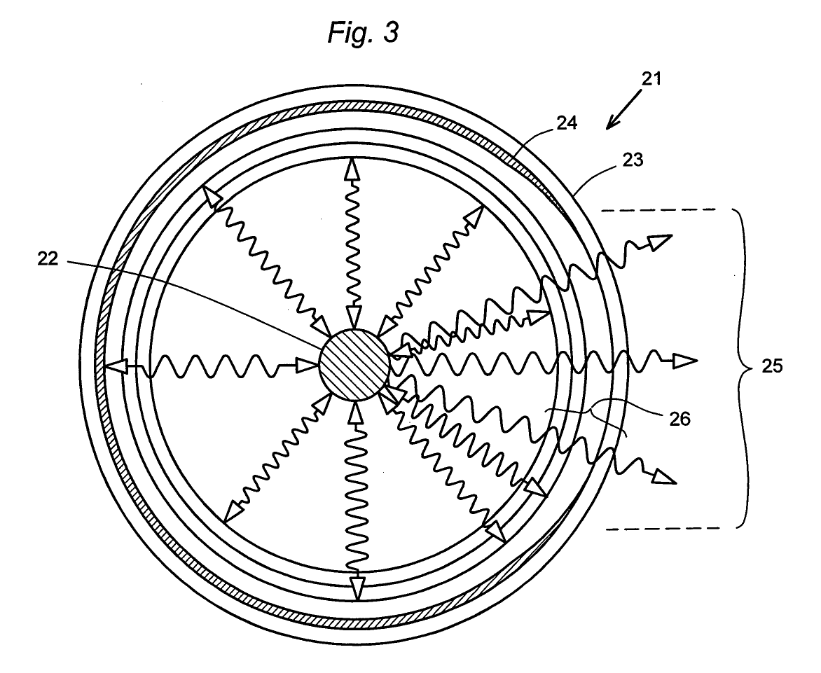

The emitter 21 of FIG. 3 is of either cylindrical or spherical configuration, again shown in cross section. The radiation source 22 in this emitter is a rod or a sphere at the center of the emitter, and the housing or envelope 23 fully surrounds the radiation source. As in the emitter of FIG. 2, the source 22 is a blackbody or gray body source activated or energized by external means (not shown) of conventional nature, the source emitting radiation at a wide range of wavelengths 16, 17, 18 in all directions. A total reflector 24 extends around a portion of the housing wall, allowing radiation to escape only through a window 25. The entire housing is coated on its interior surface with a multilayer optical coating 26 that functions in the same manner as the multilayer optical coating of the flat panel emitter of FIG. 2, allowing only radiation of a selected wavelength range to pass through the coating while reflecting the remainder back toward the source 22 with the benefit of constructive interference.

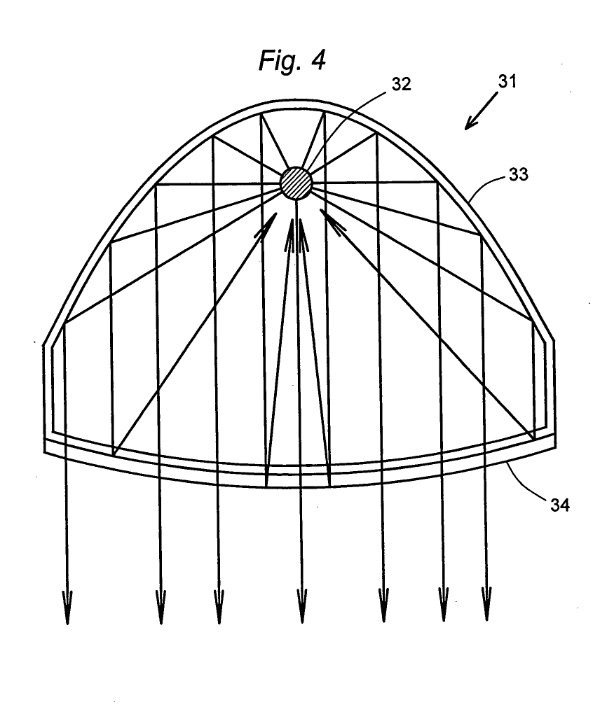

The emitter 31 of FIG. 4, like that of FIG. 2, is either a body of revolution or an elongated body with a constant cross section, the view in the Figure representing the cross section in either case. The cross section is parabolic in shape, and the radiation source 32 in this emitter is either a sphere or a rod at the focal point of the parabola, activated or energized by external means (not shown) of a conventional nature and emitting radiation over a range of wavelengths in all directions. The parabolic section 33 of the wall is a total reflector and the wall section 34 opposite the parabolic section is a transparent material coated with a multilayer optical coating functioning in the same manner as the multilayer optical coatings of the preceding Figures. The totally reflective parabolic section 33 causes all emerging radiation to occur in a single direction. If the emitter is a body of revolution, the emerging beam is of circular cross section; if the emitter is an elongated body with a constant cross section, the emerging beam is of rectangular cross section.

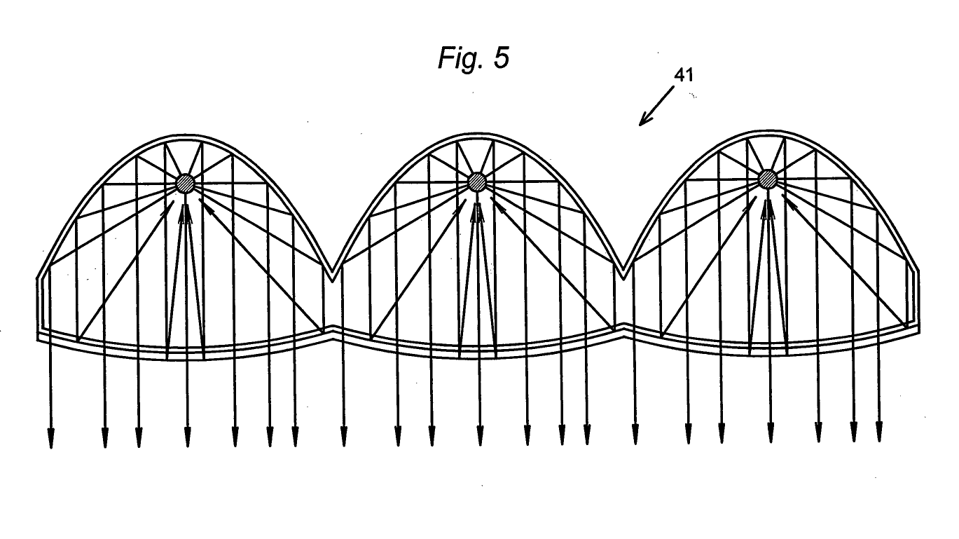

FIG. 5 depicts a series of emitters each identical to those of FIG. 4 but arranged end-to-end to provide a wider beam of emitted radiation.

Improved durability and ease of manufacturing can be achieved by forming the emitter, regardless of its shape, as a monolithic, i.e., continuous solid, body. At the core of the body is a material that is transparent either to all wavelengths or to only the desired output wavelength. The radiation source is embedded in the core, and the core is encased by a surrounding wall a section of which is a total reflector and another section a window that includes a multilayer optical coating such as that as shown in the preceding Figures and described above. The monolithic body can assume any of the shapes cited above to produce emerging beams of any of the various cross sections.

In its most generic sense, the process of this invention is used to selectively and destructively heat a target in a host in such as manner as to transform the target without any substantial transformation or harm to the host. Among the applications of the process are the dehydration of organic matter and the disinfection or disinfestation of organic matter, mammalian tissue, and living organisms. To determine the wavelength or combination of wavelengths that will most efficiently achieve these results, both the host (the organic matter, tissue or organism) and the target (moisture or pest) are evaluated and information compiled that relates to the compositions of the host and the target, and the amount and location of the target in the host.

One method for determining the most efficient wavelength or combination of wavelengths for a particular application is spectroscopy. By spectroscopic analysis, one can determine the absorption, reflectance, or transmission of a sample of host as a function of wavelength, and from this data, the chemical composition of the host can be derived. The spectrogram will contain peaks at wavelengths where high absorption occurs. Efficient results are then obtained by irradiation of the host at wavelengths at or close to those where the spikes appear.

For dehydration applications, radiation at the peak wavelengths is preferably performed for an initial period of time during which high surface temperatures develop. As the host then dries and the rate of heat conduction at the surface diminishes, continued dehydration can be achieved by shifting the wavelength of the radiation to values displaced from the peaks, preferably both above and below a particular peak, and thereby achieve greater penetration. As the treatment time progresses and the moisture content drops, power can be reduced.

For pest removal applications where dehydration is not desired, preferred wavelengths are those that coincide with less than 60% of the peaks on the host spectrogram but coincide closely with peaks associated with the pest or the solvent present in the pest.

For any applications involving the removal of water or a solvent, the depressed vapor point of the water or solvent can first be determined, and a vacuum level and optimum radiation wavelength can then be determined. With infrared radiation, for example, wavelengths between 800 and 1,000 nm will be most effective for heating below the host surface. Radiation at wavelengths between 800 and 1,000 nm will achieve good penetration with minimal surface heating, while radiation at wavelengths above 1,500 nm will provide high surface heating with minimal penetration. Hosts with high water content exhibit high heat transfer rates, and penetration is relatively unimportant in early stages of dehydration. Thus, pests and free water located near the surface of the host can be quickly flashed off with infrared radiation at appropriately selected wavelengths.

Other applications of the invention include the control or destruction of neoplastic tissue, viral infections, and any conditions whose development and proliferation are mediated by enzyme activity or by other bio-reactive substances. Thus, by first identifying substances that occur in pathogenic material and that are critical to the survival or proliferation of the material, then selectively exposing hosts, including matter and organisms, from which the pathogenic material is sought to be removed. The invention can thus be used for clinical therapy, sterilization, and in general the remediation of environmentally or physiologically unfavorable conditions. The differential absorption produced by the selective radiation achieved by the process of the present invention induces wavelength, dependent photochemical or photomechanical reactions that cause changes or transitions to occur in the target to result in selective disinfection, denaturation, disruption, or dehydration.

Protein denaturation and the destruction or transformation of polysaccharides, lipids, and liposaccharides are examples of effects that can be achieved by selective radiation in the practice of the present invention as means of destroying undesirable biological or organic matter in a host. Further examples of effective targets are peptidoglycans which occur in cell wall structures of microorganisms, porins (transport poroteins that are critical to vital cell function), liposaccharides that contain keto-deoxyoctonate, glycocalyx structures, technoic acids, autolysins, and lysozymes. The use of chitin as a target is an effective way of controlling a number of insects and microorganisms. In addition to pest control, the destruction or transformation of proteins, lipids, and polysaccharides is an effective means of controlling the flavor and texture of foods. The deactivation of enzymes in particular can preserve flavors and shelf life of foods.

Neoplastic tissue can be destroyed or rendered inactive by the process of the present invention, by deactivating the cellular endoprotease furin or by focusing on lesions and other neoplastic tissue by virtue of the distinctive characteristics of the spectra of these tissues. Articles made of synthetic polymers can be sterilized for the destruction of bacteria or any other pathogen that is spectroscopically distinct from the polymer. Examples of such articles are surgical equipment, implants, and medical devices in general, as well as oackaging for the food and beverage industry.

The foregoing is offered primarily for purposes of illustration and is not intended to limit the scope of the invention. Further variations in the materials and their configurations and arrangements will be readily apparent to those skilled in the art and can be made without departing from the spirit and scope of the invention.

1. A method for selectively and destructively heating a target in a host, said method comprising irradiating said host with electromagnetic energy at a selected wavelength at which absorption of said energy by said target exceeds absorption of said energy by other substances in said host by a sufficient absorption differential to destructively transform said target with substantially no transformation of said host, by directing radiation to said host from a spectrally selective radiation emitter comprising:
a housing,
a source of electromagnetic radiation disposed within said housing, emitting radiation having wavelengths extending across a continuous spectrum that includes said selected wavelength, and
a multi-layer optical coating arranged to intercept radiation from said source of electromagnetic radiation, adjacent layers of said coating differing in refractive index by differentials that alternate between positive and negative such that radiation at wavelengths other than a selected wavelength is reflected back toward said source of electromagnetic radiation, said layers having optical thicknesses and refractive indices selected to produce constructive interference between radiations so reflected, thereby recycling radiative emissions at said other wavelengths back to said source of electromagnetic radiation while allowing radiative emission of said selected wavelength to emerge from said housing.

2. The method of claim 1 wherein said source of electromagnetic radiation is a gray body emitter.

3. The method of claim 1 wherein said source of electromagnetic radiation is a member selected from the group consisting of an incandescent bulb, a heat lamp, a resistance heater, a gas ceramic emitter, and an electric ceramic emitter.

4. The method of claim 1 wherein adjacent layers of said coating differ in refractive index by a refractive index ratio of about 1.3 or higher.

5. The method of claim 1 wherein adjacent layers of said coating alternate between layers have a refractive index greater than or equal to 2.1 and layers have a refractive index less than or equal to 1.8.

6. The method of claim 1 wherein adjacent layers of said coating alternate between layers have a refractive index of about 2.1 to about 2.7 and layers have a refractive index of about 1.3 to about 1.8.

7. The method of claim 1 wherein said multi-layer optical coating consists of from 4 to 50 pairs of adjacent layers, the layers in each pair different in refractive index by said differentials.

8. The method of claim 1 wherein said multi-layer optical coating consists of from 5 to 20 pairs of adjacent layers, the layers in each pair different in refractive index by said differentials.

9. The method of claim 1 wherein said multi-layer optical coating comprises first and second segments, said first segment reflecting radiation at wavelengths above said selected wavelength and said second segment reflecting radiation at wavelengths below said selected wavelength.

10. The method of claim 1 wherein said multi-layer optical coating comprises first, second and third segments, said first and second segments reflecting radiation at wavelengths above and below a first selected wavelength, and said second and third segments reflecting radiation at wavelengths above and below a second selected wavelength.

11. The method of claim 1 wherein said spectrally selective radiation emitter further comprises a total reflector disposed within said housing.

12. The method of claim 11 wherein said total reflector has a parabolic cross section and said source of electromagnetic radiation is positioned at the focal point of said parabolic cross section.

13. The method of claim 1 wherein said host is organic matter and said target is a pest infesting said organic matter.

14. The method of claim 1 wherein said host is moisture-containing organic matter and said target is moisture.

15. The method of claim 1 wherein said host is a living organism and said target is neoplastic tissue.

16. The method of claim 1 wherein said host is living tissue and said taget is an enzyme.

17. The method of claim 1 wherein said host is a body of a member selected from the group consisting of polyethylene, polystyrene, and polypropylene, and said target is glucose.

18. The method of claim 1 wherein said host is a body of silicone and said target is proteinaceous matter.

19. The method of claim 1 wherein said target is a bio-reactive substance selected from the group consisting of RNases, DNases, pyrogens, and nucleic acids.

20. The method of claim 1 wherein said host is mammalian tissue infected with a microorganism, and said target is said microorganism.

21. The method of claim 1 wherein said host is a foodstuff and said target is foreign matter in said foodstuff.

22. A spectrally selective radiation emitter comprising:
a housing,
a hot body source of electromagnetic radiation disposed within said housing, emitting radiation having wavelengths extending across a continuous spectrum that includes said selected wavelength, and
a multi-layer optical coating arranged to intercept radiation from said source, adjacent layers of said coating differing in refractive index by differentials that alternate between positive and negative such that radiation at wavelengths other than a selected wavelength is reflected back toward said source, said layers having optical thicknesses and refractive indices selected to produce constructive interference between radiations so reflected, thereby recycling radiative emissions at said other wavelengths back to said solid body source while allowing radiative emission of said selected wavelength to emerge from said housing.

23. The spectrally selective radiation emitter of claim 22 wherein said source of electromagnetic radiation is a gray body emitter.

24. The spectrally selective radiation emitter of claim 22 wherein said source of electromagnetic radiation is an infrared light source.

25. The spectrally selective radiation emitter of claim 22 wherein said source of electromagnetic radiation is a hot body emitter.

26. The spectrally selective radiation emitter of claim 22 wherein said multi-layer optical coating consists of from 4 to 50 pairs of adjacent layers.

27. The spectrally selective radiation emitter of claim 22 wherein said multi-layer optical coating consists of from 5 to 20 pairs of adjacent layers.

28. The spectrally selective radiation emitter of claim 22 wherein said multi-layer optical coating comprises first and second segments, said first segment reflecting radiation at wavelengths above said selected wavelength and said second segment reflecting radiation at wavelengths below said selected wavelength.

29. The spectrally selective radiation emitter of claim 22 wherein said multi-layer optical coating comprises first, second and third segments, said first and second segments reflecting radiation at wavelengths above and below a first selected wavelength, and said second and third segments reflecting radiation at wavelengths above and below a second selected wavelength.

30. The spectrally selective radiation emitter of claim 22 further comprising a total reflector disposed within said housing.

31. The spectrally selective radiation emitter of claim 30 wherein said total reflector has a parabolic cross section and said source of electromagnetic radiation is positioned at the focal point of said parabolic cross section.
